(19) United States
(12) Patent Application Publication
(10) Pub. No.: US 2023/0381273 A1
(43) Pub. Date: Nov. 30, 2023

(54) NANOPARTICLE IN WHICH ANTIBACTERIAL PEPTIDE IS ENCAPSULATED WITH CHITOSAN AND USE THEREOF

(71) Applicant: NATIONAL MARINE BIODIVERSITY INSTITUTE OF KOREA, Seocheon-gun, (KR)

(72) Inventors: Ilson Whang, Seocheon-gun (KR); De Zoysa Mahanama, Yuseong-gu (KR); Nikapitiya Chamilani Lalanthi, Yuseong-gu (KR); Eriyawala Hewage Thimira Thulshan Jayathilaka, Yuseong-gu (KR)

(21) Appl. No.: 18/298,798

(22) Filed: Apr. 11, 2023

(30) Foreign Application Priority Data
Apr. 11, 2022 (KR) 10-2022-0044711
Oct. 19, 2022 (KR) 10-2022-0135153

(51) Int. Cl. A61K 38/17; A61K 9/51; A61P 31/04

(52) U.S. Cl. CPC A61K 38/1767; A61P 31/04; A61K 9/5161

(57) ABSTRACT

In the present disclosure, octominin-chitosan nanoparticle (octominin-CNP) encapsulated with octominin was prepared, and its antibacterial, antifungal and anti-biofilm activities against A. baumannii and C. albicans were confirmed. The optimal mixing ratio thereof is derived, the octominin-CNP alleviates the cytotoxicity of octominin itself and causes a greater morphological change on the cell surface of A. baumannii and C. albicans than octominin itself, showing excellent antibacterial and antifungal activity as well as excellent biofilm formation inhibition and eradication effects, so that the octominin-CNP may be usefully used for antibacterial or biofilm formation inhibition.

### STATEMENT REGARDING ELECTRONIC FILING OF A SEQUENCE LISTING

A Sequence Listing in XML format, submitted under 37 C.F.R. § 1.821, entitled 1547-11_ST26.XML, 2,162 bytes in size, generated on Apr. 11, 2023, and filed electronically, is provided in lieu of a paper copy. This Sequence Listing is hereby incorporated by reference into the specification for its disclosures.

### CROSS-REFERENCE TO RELATED APPLICATIONS

This application is based on and claims priority from Korean Patent Application No. 10-2022-0044711, filed on Apr. 11, 2022, and Korean Patent Application No. 10-2022-0135153, filed on Oct. 19, 2022, the disclosures of each of which are incorporated herein by reference in its entirety.

### FIELD OF THE INVENTION

The present disclosure relates to nanoparticles in which antibacterial peptides are encapsulated with chitosan and their uses based on their antibacterial, antifungal and anti-biofilm activity against Acinetobacter baumannii and Candida albicans.

### BACKGROUND OF THE INVENTION

Antibiotics are generally substances that have an antibacterial action against bacteria, specifically, substances that have an excellent antibacterial action by inhibiting a system in which bacteria synthesize cell walls or proteins, or substances produced from such substances.

Until now, numerous antibiotics have been isolated from natural products or synthesized organically, making it possible to treat many diseases or infections. However, due to excessive misuse of antibiotics worldwide, bacteria resistant to existing antibiotics are emerging, and the resistance rate is increasing. In addition, the diversified infection routes accelerate the spread of resistant bacteria, emerging as a serious social problem.

Meanwhile, Acinetobacter baumannii (A. baumannii), a pathogenic microorganism, is a gram-negative aerobic bacterium and is an important cause of nosocomial infections in many hospitals. In particular, recently, A. baumannii with reduced antibacterial effect against aminoglycoside, cephalosporin, fluoroquinolone, beta-lactamase inhibitors, and carbapenem has also been reported.

Candida albicans (C. albicans) is one of the most common fungal species parasitic on humans, which settles without subjective symptoms in the gastrointestinal tract and genitourinary tract of healthy people and is an opportunistic pathogen that may cause infection in certain pathological and physiological conditions, including diabetes, pregnancy, steroid chemotherapy and administration of broad-spectrum antibiotics, and acquired immunodeficiency syndrome. Some pathogenic strains of C. albicans exhibit multidrug resistance and/or reduced antibiotic efficacy to the currently used antifungal agents fluconazole, itraconazole, nystatin, caspofungin, ketoconazole, flucytosine and amphotericin B.

Therefore, there is a need for the development of new antibiotics with excellent antibacterial properties against A. baumannii and C. albicans.

In this regard, generally, antimicrobial peptides (AMPs) having short peptides with less than 50 amino acid residues are evolutionarily conserved for host defense against pathogenic microorganisms and can be considered as “natural antibiotics.”

The mechanism by which antimicrobial peptides act on pathogens is very different from that of existing antibiotics. They mainly bind to the cell surface of microorganisms and then form pores in the cell membrane, disrupting the normal permeability characteristics of the cell membrane to rapidly kill pathogens. Due to these characteristics, antimicrobial peptides are attracting attention as they can be used as novel antibiotics that may control antibiotic-resistant bacteria.

However, there is a need for more research on a technology capable of enhancing the antibacterial activity of the AMP while also increasing safety.

### SUMMARY OF THE INVENTION

While seeking a method for increasing the safety and antibacterial activity of antimicrobial peptides, the inventors of the present disclosure prepared nanoparticles encapsulated with chitosan and identified the effect of increasing the antibacterial and antifungal activity against A. baumannii and C. albicans and reducing the cytotoxicity of the antibacterial peptide itself, thereby completing the present disclosure.

Accordingly, the object of the present disclosure is to provide a method for inhibiting infection of Acinetobacter sp. strains.

Another object of the present disclosure is to provide a method for inhibiting biofilm.

Still another object of the present disclosure is to provide a method for preventing or treating infection with Acinetobacter sp. strain.

Still another object of the present disclosure is to provide a method for inhibiting bacteria or fungi.

Still another object of the present disclosure is to provide a method for inhibiting biofilm.

Still another object of the present disclosure is to provide a method for preventing or treating a disease caused by infection with any one strain selected from the group consisting of Acinetobacter baumannii and Candida albicans.

Still another object of the present disclosure is to provide a method for preparing antibacterial or antifungal nanoparticles.

In order to achieve the above objects, the present disclosure provides a method for inhibiting infection of an Acinetobacter sp. strain, the method including the step of treating an individual with an antimicrobial peptide consisting of the amino acid sequence represented by SEQ ID NO: 1 or a fragment thereof.

In order to achieve another object, the present disclosure provides a method for inhibiting a biofilm, the method including the step of treating an antimicrobial peptide consisting of the amino acid sequence represented by SEQ ID NO: 1 or a fragment thereof.

In order to achieve still another object, the present disclosure provides a method for preventing or treating an infectious disease by an Acinetobacter sp. strain, the method including the step of treating an individual with an antimicrobial peptide consisting of the amino acid sequence represented by SEQ ID NO: 1 or a fragment thereof.

In order to achieve still another object, the present disclosure provides a method for inhibiting bacteria or fungi, the method including the step of treating an individual with a nanoparticle in which an antibacterial peptide consisting of the amino acid sequence represented by SEQ ID NO: 1 or a fragment thereof is encapsulated with chitosan.

In order to achieve still another object, the present disclosure provides a method for inhibiting a biofilm, the method including the step of treating an individual with a nanoparticle in which an antibacterial peptide consisting of the amino acid sequence represented by SEQ ID NO: 1 or a fragment thereof is encapsulated with chitosan.

In order to achieve still another object, the present disclosure provides a method for preventing or treating a disease caused by infection with any one strain selected from the group consisting of Acinetobacter baumannii and Candida albicans, the method including the step of treating an individual with a nanoparticle in which an antibacterial peptide consisting of the amino acid sequence represented by SEQ ID NO: 1 or a fragment thereof is encapsulated with chitosan.

In order to achieve still another object, the present disclosure provides a method for producing an antibacterial or antifungal nanoparticle in which an antibacterial peptide consisting of the amino acid sequence represented by SEQ ID NO: 1 or a fragment thereof is encapsulated with chitosan, the method including the step of mixing an antibacterial peptide consisting of the amino acid sequence represented by SEQ ID NO: 1 or a fragment thereof, chitosan and a chitosan derivative.

In the present disclosure, octominin-chitosan nanoparticle (octominin-CNP) encapsulated with octominin was prepared, and its antibacterial, antifungal and anti-biofilm activities against A. baumannii and C. albicans were confirmed. The octominin-CNP alleviates the cytotoxicity of octominin itself and causes a greater morphological change on the cell surface of A. baumannii and C. albicans than octominin itself, showing excellent antibacterial and antifungal activity as well as excellent biofilm formation inhibition and eradication effects, so that the octominin-CNP may be usefully used for antibacterial or biofilm formation inhibition.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 shows a result of confirming release kinetics over time of nanoparticles (octominin-CNP) in which an antimicrobial peptide (octominin) according to the present disclosure is encapsulated with chitosan.

FIG. 2 shows a result of confirming the morphological characteristics of octominin-CNP according to the present disclosure and the control group (nanoparticles in which octominin is not encapsulated with chitosan (CNP)). FIG. 2, panel A shows a transmission electron microscopy (TEM) image of CNP, and FIG. 2, panel B shows a TEM image of octominin-CNP. FIG. 2, panel C shows an emission scanning electron microscopy (FE-SEM) image of CNP, and FIG. 2, panel D shows a FE-SEM image of octominin-CNP. In FIG. 2, a black bar means 200 nm and a white bar means 1 μm.

FIG. 3 shows a result of confirming the cytotoxicity of octominin-CNP according to the present disclosure and free octominin to HEK 293 cells.

FIG. 4 shows a result of time-kill kinetic activity according to the treatment of octominin-CNP according to the present disclosure and free octominin to A. baumannii and C. albicans at various concentrations in which FIG. 4, panel A shows the result for C. albicans, and FIG. 4, panel B shows the result for A. baumannii.

FIG. 5 shows a result of confirming the ultra-structural change of the respective strains after treating A. baumannii and C. albicans with octominin-CNP according to the present disclosure or octominin, FIG. 5, panel A is a negative control group treated with PBS to C. albicans, FIG. 5, panel B is an experimental group treated with octominin with MIC (50 μg/mL) to C. albicans, FIG. 5, panel C is an experimental group treated with octominin with MFC (200 μg/mL) to C. albicans, FIG. 5, panel D is an experimental group treated with CNP to C. albicans, FIG. 5, panel E is an experimental group treated with octominin-CNP with MIC (in an octominin concentration of 50 μg/mL) to C. albicans, and FIG. 5, panel F is an experimental group treated with octominin-CNP with MFC (in an octominin concentration of 200 μg/mL) to C. albicans. FIG. 5, panel G is a negative control group treated with PBS to A. baumannii, FIG. 5, panel H is an experimental group treated with octominin with MIC (5 μg/mL) to A. baumannii, FIG. 5, panel I is an experimental group treated with octominin with MBC (10 μg/mL) to A. baumannii, FIG. 5, panel J is an experimental group treated with CNP to A. baumannii, FIG. 5, panel K is an experimental group treated with octominin-CNP with MIC (in an octominin concentration of 5μg/mL) to A. baumannii, and FIG. 5, panel L is an experimental group treated with octominin-CNP with MBC (in an octominin concentration of 10 μg/mL) to A. baumannii. In FIG. 5, panel A to FIG. 5, panel F, a white bar means 2 μm, and in FIG. 5G to FIG. 5L, a white bar means 200 nm.

FIG. 6 is a result of confirming the cell membrane permeability change after treating C. albicans with octominin-CNP according to the present disclosure or octominin.

FIG. 7 is a result of confirming the cell membrane permeability change after treating A. baumannii with octominin-CNP according to the present disclosure or octominin.

FIG. 8 is a result of confirming the ROS (reactive oxygen species) generating ability after treating C. albicans with octominin-CNP according to the present disclosure or octominin.

FIG. 9 is a result confirming the ROS (reactive oxygen species) generating ability after treating A. baumannii with octominin-CNP according to the present disclosure or octominin.

FIG. 10 is a result of confirming the anti-biofilm activity of A. baumannii and C. albicans treated with octominin-CNP according to the present disclosure and free octominin at various concentrations, FIG. 10, panel A is the result of confirming the biofilm formation inhibitory effect against C. albicans, FIG. 10, panel B is the result of confirming the biofilm formation inhibitory effect against A. baumannii, FIG. 10, panel C is the result of confirming the eradication effect on the biofilm already formed by C. albicans, and FIG. 10, panel D is the result of confirming the eradication effect on the biofilm already formed by of A. baumannii.

### DETAILED DESCRIPTION OF THE INVENTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

Hereinafter, the present disclosure is described in detail.

The inventors of the present disclosure confirmed the excellent antibacterial activity of the antimicrobial peptide against a specific strain. Further, while seeking a method to increase the safety and antibacterial activity of the antimicrobial peptide, nanoparticles in which the antimicrobial peptide was encapsulated with chitosan were prepared to confirm its cytotoxicity, antibacterial, antifungal activity and anti-biofilm activity against infectious strains, completing the present disclosure.

Therefore, the present disclosure provides a method for inhibiting infection of an Acinetobacter sp. strain, the method including the step of treating an individual with an antimicrobial peptide consisting of the amino acid sequence represented by SEQ ID NO: 1 or a fragment thereof.

(SEQ ID NO: 1)
GWLIRGAIHAGKAIHGLIHRRRH

The antibacterial peptide consisting of the amino acid sequence represented by SEQ ID NO: 1 is a novel octopus minor-derived antimicrobial peptide (AMP) discovered by the inventors of the present disclosure, which is named octominin.

The antimicrobial peptide according to the present disclosure may include an amino acid sequence in which one or more amino acid residues are conservatively substituted in 5 or more, preferably 7 or more, more preferably 10 or more, or most preferably 18 or more contiguous amino acid sequences of the amino acid sequence represented by SEQ ID NO: 1. Conservative amino acid substitutions may include substitutions with amino acid residues that have little or no effect on the size, polarity, hydrophobicity, or hydrophilicity of the amino acid residue.

The antimicrobial peptide according to the present disclosure may be characterized by having an amino acid sequence having 90% or more, preferably 95% or more, more preferably 98% or more homology with the amino acid sequence represented by SEQ ID NO: 1 or 5 or more, preferably 7 or more, more preferably 10 or more, or most preferably 18 or more contiguous amino acid sequences of the amino acid sequence represented by SEQ ID NO: 1.

According to an embodiment of the present disclosure, the antimicrobial peptide consisting of the amino acid sequence represented by SEQ ID NO: 1 showed excellent antibacterial activity against Acinetobacter sp. strain, particularly Acinetobacter baumannii (A. baumannii).

Further, according to another embodiment of the present disclosure, it was confirmed that the antimicrobial peptide consisting of the amino acid sequence represented by SEQ ID NO: 1 causes ultrastructural cell wall modification of A. baumannii, thereby inducing resistance to A. baumannii. Further, through still another embodiment, it was confirmed by propidium iodide uptake analysis that the antibacterial peptide consisting of the amino acid sequence represented by SEQ ID NO: 1 penetrates A. baumannii cells to cause cell membrane loss and cell death. Further, it was also confirmed that the production of reactive oxygen species in A. baumannii cells was increased.

In the present disclosure, “antibacterial”, “antibacterial activity” or “antifungal activity” means a property that resists microorganisms such as bacteria and fungi, and more specifically, it means the property of antibiotics to inhibit the growth or proliferation of bacteria.

The method for inhibiting infection of the Acinetobacter sp. strain of the present disclosure may be performed by treating an individual with an antimicrobial composition including an antimicrobial peptide consisting of an amino acid sequence represented by SEQ ID NO: 1 or a fragment thereof.

In the present disclosure, the “antibacterial composition” is a composition having an activity to inhibit the growth of microorganisms such as bacteria and fungi and may include all forms used in various fields requiring antibacterial effects, for example, may be in the form of pharmaceuticals, quasi-drugs, food additives, feed additives, or the like. Specifically, they may be used in medicine for purposes such as antibiotics and antifouling agents, in food for preservative or antibacterial purposes, in agriculture for the purpose of antibacterial, sterilization and disinfection, or in cosmetics and household goods for products directly related to microorganisms such as anti-dandruff, anti-athlete, anti-armpit, anti-acne, etc. or for the purpose of preservative, antibacterial or sterilization, such as cleaning detergent or dishwashing detergent, but is not limited to these purposes. In the present disclosure, the antibacterial composition means a composition with the activity of inducing inhibition of the growth or death of A. baumannii based on the antimicrobial activity against A. baumannii of the antimicrobial peptide consisting of the amino acid sequence represented by SEQ ID NO: 1 as a component.

In the present disclosure, the term “individual” includes both biological and non-biological objects and is not limited as long as it is an individual that requires the death of Acinetobacter sp. strains.

Further, according to still another embodiment of the present disclosure, the antimicrobial peptide consisting of the amino acid sequence represented by SEQ ID NO: 1 not only inhibits the formation of a biofilm of A. baumannii, but also eradicates the existing biofilm formed by A. baumannii, thereby showing excellent anti-biofilm effect.

Thus, the present disclosure provides a method for inhibiting a biofilm, the method including the step of treating an antimicrobial peptide consisting of the amino acid sequence represented by SEQ ID NO: 1 or a fragment thereof.

In the present disclosure, the “biofilm” is a film that appears at a part infected or attached to microorganisms. It is also called a biofilm as a film that is surrounded by a polymer substrate and forms a microbial complex produced by microorganisms. The biofilm is important for the survival of microorganisms because it not only serves as a protective film for microorganisms, but also allows different cells to meet and share metabolism and acquires beneficial properties through gene transfer. That is, the biofilm is an aggregate of various bacteria as well as serving as a protective film for the bacterial population, so that it is a direct or indirect cause of various diseases caused by bacteria.

The method of inhibiting a biofilm of the present disclosure may be performed by treating an individual with a composition for inhibiting a biofilm, including an antimicrobial peptide consisting of the amino acid sequence represented by SEQ ID NO: 1 or a fragment thereof.

In the present disclosure, the “individual” includes all biological or non-biological objects on which a biofilm has been formed or is expected to be formed by A. baumannii.

The composition for inhibiting biofilm of the present disclosure may inhibit biofilm formation by A. baumannii or eradicate previously formed biofilm.

Further, the present disclosure provides a method for preventing or treating an infectious disease by an Acinetobacter sp. strain, the method including the step of treating an individual with an antimicrobial peptide consisting of the amino acid sequence represented by SEQ ID NO: 1 or a fragment thereof based on A. baumannii antibacterial activity of an antimicrobial peptide consisting of the amino acid sequence represented by SEQ ID NO: 1.

The method for preventing or treating an infectious disease by an Acinetobacter sp. strain of the present disclosure may be performed by administering to an individual a pharmaceutical composition including an antimicrobial peptide consisting of the amino acid sequence represented by SEQ ID NO: 1 or a fragment thereof.

In the present disclosure, the term “individual” refers to mammals such as horses, sheep, pigs, goats, and dogs, including humans, birds, fish, crustaceans, reptiles, amphibians, etc., which may show a therapeutic effect on Acinetobacter infectious diseases, preferably a human.

The pharmaceutical composition of the present disclosure is preferably administered orally or parenterally.

Oral administration includes intraoral administration, and the pharmaceutical composition of the present disclosure may be administered orally in any orally acceptable form, including but not limited to pills, dragees, capsules, liquids, gels, syrups, slurries, and suspensions.

Oral tablets include lactose and corn starch as carriers commonly used. A lubricant such as magnesium stearate is also typically added. For oral administration in capsule form, useful diluents include lactose and dried corn starch. For oral administration in an aqueous suspension, the active ingredient is combined with emulsifying and suspending agents. If desired, sweetening and/or flavoring and/or coloring agents may be added.

A pharmaceutical composition for oral administration may be prepared by mixing the active ingredient with a solid excipient and may be prepared in granule form for preparation in the form of tablets or dragees. Suitable excipients may include sugar forms such as lactose, sucrose, mannitol and sorbitol, or starch from corn, wheat flour, rice, potato or other plants, cellulose such as methyl cellulose, hydroxypropylmethyl-cellulose or sodium carboxymethylcellulose, carbohydrates such as gums including gum arabic and tagacanthus, or protein fillers such as gelatin and collagen. If desired, disintegrants or solubilizers in the form of cross-linked polyvinylpyrrolidone, agar and their respective salts such as alginic acid or sodium alginate may be added.

In the present disclosure, “parenteral” includes subcutaneous, intradermal, intravenous, intramuscular, intraarticular, intracapsular, intrasternal, intrathecal, intralesional and intracranial injection or infusion techniques.

The pharmaceutical composition of the present disclosure may be in the sterile injectable preparation as a sterile injectable aqueous or oleaginous suspension. This suspension may be formulated according to techniques known in the art using suitable dispersing or wetting agents (e.g., Tween 80) and suspending agents. The sterile injectable preparation may also be a sterile injectable solution or suspension in a non-toxic parenterally acceptable diluent or solvent (e.g., as a solution in 1,3 -butanediol). Vehicles and solvents that may be acceptably employed include mannitol, water, Ringer's solution and isotonic sodium chloride solution. Further, sterile non-volatile oils are usually employed as a solvent or suspending medium. For this purpose, any bland non-volatile oil may be employed including synthetic mono- or diglycerides. Fatty acids such as oleic acid and its glyceride derivatives are useful in injectable formulations as are pharmaceutically acceptable natural oils (e.g., olive oil or castor oil), especially polyoxyethylated ones thereof. Further, for parenteral administration, the pharmaceutical composition of the present disclosure may be prepared as an aqueous solution. Preferably, a physically appropriate buffer solution such as Hank's solution, Ringer's solution or physically buffered saline may be used. Aqueous injection suspensions may contain substances which increase the viscosity of the suspension, such as sodium carboxymethyl cellulose, sorbitol or dextran. In addition, suspensions of the active ingredient may be prepared as suitable oily injection suspensions. Suitable lipophilic solvents or carriers include fatty acids such as sesame oil or synthetic fatty acid esters such as ethyl oleate, triglycerides or liposomes. Polycationic amino polymers may also be used as carriers. Optionally, the suspension may use suitable stabilizers or agents to increase the solubility of the compounds and to prepare highly concentrated solutions.

The pharmaceutical composition of the present disclosure may also be administered in the form of a suppository for rectal administration. These compositions may be prepared by mixing the antimicrobial peptides of the present disclosure or fragments thereof with a suitable nonirritating excipient that is solid at room temperature but liquid at rectal temperature. Such materials include, but are not limited to, cocoa butter, beeswax, and polyethylene glycol.

When the pharmaceutical composition of the present disclosure is applied topically to the skin, the pharmaceutical composition should be formulated as a suitable ointment containing the active ingredient suspended or dissolved in a carrier. Carriers for topical administration of the compounds of the present disclosure include, but are not limited to, mineral oil, liquid paraffin, white petrolatum, propylene glycol, polyoxyethylene, polyoxypropylene compound, emulsifying wax and water. Further, the pharmaceutical composition of the present disclosure may be formulated as a suitable lotion or cream containing the active compound suspended or dissolved in a carrier. Suitable carriers include, but are not limited to, mineral oil, sorbitan monostearate, polysorbate 60, cetyl ester wax, cetearyl alcohol, 2-octyldodecanol, benzyl alcohol and water. The pharmaceutical composition of the present disclosure may also be topically applied to the lower intestinal tract by rectal suppository and also as a suitable enema. Topically applied transdermal patches are also included in the present disclosure.

The pharmaceutical composition of the present disclosure may be administered by intranasal aerosol or inhalation. Such compositions are prepared according to techniques well known in the field of medicine and may be prepared as a solution in saline using benzyl alcohol or other suitable preservatives, absorption enhancers to increase bioavailability, fluorocarbons and/or other solubilizing or dispersing agents known in the art.

The specific effective amount for a specific patient depends on a number of factors, including the activity of the specific compound used, age, body weight, general health, gender, diet, time of administration, route of administration, rate of excretion, the drug combination and the severity of the particular disease being prevented or treated.

To increase or enhance the prevention, alleviation or treatment effect against Acinetobacter sp. strain infection, the pharmaceutical composition of the present disclosure may further include any compound or natural extract known to have an effect of preventing, ameliorating, or treating an infection with Acinetobacter sp. whose safety has already been verified in addition to the antibacterial peptide consisting of the amino acid sequence represented by SEQ ID NO: 1 or fragment thereof.

Further, the present disclosure provides a food composition including an antimicrobial peptide consisting of the amino acid sequence represented by SEQ ID NO: 1 or a fragment thereof.

The food composition according to the present disclosure includes all forms such as functional food, nutritional supplement, health food, health supplement and food additives. The type of food composition may be formulated in any one form selected from the group consisting of powders, tablets, capsules, pills and liquids according to conventional methods known in the art, but is not limited thereto. It may be prepared in various forms using methods known in the art.

For example, as a health food, an antibacterial peptide consisting of the amino acid sequence represented by SEQ ID NO: 1 of the present disclosure or a fragment thereof may be ingested by being granulated, encapsulated, and powdered or consumed by being prepared in the form of tea, juice, and drink. Further, an antibacterial peptide consisting of the amino acid sequence represented by SEQ ID NO: 1 or a fragment thereof is mixed with known substances or active ingredients known to have activity in preventing, alleviating or treating infections caused by the Acinetobacter sp. strain to be prepared in the form of a composition.

Further, the functional food may be prepared by adding an antibacterial peptide consisting of the amino acid sequence represented by SEQ ID NO: 1 or a fragment thereof to beverages (including alcoholic beverages), fruits and their processed foods (e.g., canned fruits, bottled products, jams, marmalades, etc.), fish, meat and their processed foods (e.g., ham, sausage corned beef, etc.), bread and noodles (e.g., udon, buckwheat noodles, ramen, spaghetti, macaroni, etc.), fruit juice, various drinks, cookies, taffy, dairy products (e.g., butter, cheese, etc.), edible vegetable oil, margarine, vegetable protein, retort food, frozen food, and various seasonings (e.g., soybean paste, soy sauce, sauce, etc.).

Further, the food composition of the present disclosure may include conventional food additives, and whether or not it is suitable as a “food additive” is determined by the standards and criteria for the item in accordance with the general rules of the Food Additive Code and general test methods approved by the Ministry of Food and Drug Safety, unless otherwise specified. Items listed in the “Food Additive Code” may include, for example, chemical compounds such as ketones, glycine, potassium citrate, nicotinic acid, and cinnamic acid, natural additives such as persimmon pigment, licorice extract, crystalline cellulose, sorghum pigment, guar gum, and mixed preparations such as sodium L-glutamate preparations, noodle-added alkali preparations, preservative preparations, and tar color preparations.

In the food composition of the present disclosure, the antimicrobial peptide consisting of the amino acid sequence represented by SEQ ID NO: 1 or a fragment thereof may be preferably included in an amount of 0.00001% by weight to 50% by weight compared to the food composition. If the content is less than 0.00001% by weight, the effect is insufficient, and if it exceeds 50% by weight, the increase in effect compared to the amount used is insignificant, which is uneconomical.

Further, in order to use the antibacterial peptide consisting of the amino acid sequence represented by SEQ ID NO: 1 or fragment thereof of the present disclosure in the form of a food additive, it maty be prepared and used in the form of tablets, capsules, powders, granules, liquids, pills, etc.

When the composition of the present disclosure is prepared as a beverage, it may contain various flavoring agents or natural carbohydrates as additional components, like conventional beverages. The aforementioned natural carbohydrates may include monosaccharides such as glucose and fructose, disaccharides such as maltose and sucrose, natural sweeteners such as dextrin and cyclodextrin, and synthetic sweeteners such as saccharin and aspartame. The proportion of the natural carbohydrate is generally about 0.01 g to 10 g, preferably about 0.01 g to 0.1 g per 100 ml of the composition of the present disclosure.

In addition to the above, the composition of the present disclosure may include various nutrients, vitamins, electrolytes, flavors, colorants, pectic acid and its salts, alginic acid and its salts, organic acids, protective colloidal thickeners, pH adjusters, stabilizers, preservatives, glycerin, alcohol, carbonation agents used in carbonated beverages, and the like. In addition, the composition of the present disclosure may include fruit flesh for preparing natural fruit juice, fruit juice beverages, and vegetable beverages. These components may be used independently or in combination. The proportion of these additives is not critical, but is generally selected from the range of 0.01 parts by weight to 0.1 parts by weight per 100 parts by weight of the composition of the present disclosure.

Further, the present disclosure provides a cosmetic composition including an antimicrobial peptide consisting of the amino acid sequence represented by SEQ ID NO: 1 or a fragment thereof.

Ingredients included in the cosmetic composition of the present disclosure may include ingredients commonly used in cosmetic compositions in addition to the antibacterial peptide consisting of the amino acid sequence represented by SEQ ID NO: 1 or a fragment thereof, for example, conventional adjuvants and carriers such as stabilizers, solubilizers, vitamins, pigments and flavors.

The cosmetic composition of the present disclosure may be prepared in any formulation conventionally prepared in the art, and examples thereof may include emulsions, creams, face lotions, packs, foundations, lotions, beauty essences, and hair cosmetics.

Specifically, the cosmetic composition of the present disclosure includes formulations of skin lotions, skin softeners, skin toners, astringents, lotions, milk lotions, moisture lotions, nutrient lotions, massage creams, nutrient creams, moisture creams, hand creams, foundations, essences, nutrient essences, packs, soaps, cleansing foams, cleansing lotions, cleansing creams, body lotions, and body cleansers.

More preferably, it may be a formulation of hair tonic, hair cream, hair lotion, hair shampoo, hair rinse, hair conditioner, hair spray, hair aerosol, pomade, powder gel, hair pack, hair treatment or eyelash nutrition, but is limited thereto.

When the formulation of the present disclosure is a paste, cream or gel, the carrier component may include animal fiber, vegetable fiber, wax, paraffin, starch, tracanth, cellulose derivative, polyethylene glycol, silicone, bentonite, silica, talc or zinc oxide.

When the formulation of the present disclosure is a powder or spray, the carrier component may include lactose, talc, silica, aluminum hydroxide, calcium silicate or polyamide powder, and in particular, when the formulation of the present disclosure is a spray, the carrier component may additionally include a propellant such as chlorofluorohydrocarbon, propane/butane or dimethyl ether.

When the formulation of the present disclosure is a solution or emulsion, the carrier component may include a solvent, solvating agent or emulsifying agent, such as water, ethanol, isopropanol, ethyl carbonate, ethyl acetate, benzyl alcohol, benzyl benzoate, propylene glycol, 1,3-butyl glycol oil, glycerol aliphatic esters, fatty acid esters of polyethylene glycol or sorbitan.

When the formulation of the present disclosure is a suspension, the carrier component may include a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar or tracanth, and the like.

When the formulation of the present disclosure is a surfactant-containing cleanser, the carrier component may include aliphatic alcohol sulfate, aliphatic alcohol ether sulfate, sulfosuccinic acid monoester, isethionate, imidazolinium derivative, methyl taurate, sarcosinate, fatty acid amide ether sulfate, alkylamidobetaine, fatty alcohol, fatty acid glyceride, fatty acid diethanolamide, vegetable oil, linolin derivative, ethoxylated glycerol fatty acid ester, or the like.

In addition to the above active components, the cosmetic composition of the present disclosure may further include one or more components that help improve skin conditions, including components for preventing, alleviating, or treating infections caused by Acinetobacter sp., which have identical or similar functions. The component may include hyaluronic acid, butylene glycol, glycerin, amino acid, trehalose, kojic acid and its derivatives, arbutin, ascorbic acid and its derivatives, hydroquinone and its derivatives, resorcinol, 2,7-dinitroindazole, adenosine, retinol, retinyl palmitate, polyethoxylated retinamide, yeast, dipeptide, palmitoyl oligopeptide & palmitoyl tripeptide-7, acetyl hexapeptide, epidermal growth factor (EGF), or plant extracts such as tangerine, rice, licorice, shea butter, aloe vera, coconut, olive, and avocado, but is not limited thereto.

In the present disclosure, the antimicrobial peptide consisting of the amino acid sequence represented by SEQ ID NO: 1 or a fragment thereof may be added in an amount of 0.001% by weight to 50.0% by weight based on the total weight of the cosmetic composition, preferably 0.005% by weight to 10.0% by weight. If the content is less than 0.001% by weight, it is difficult to expect a substantial preventive or improving effect, and if it exceeds 50% by weight, manufacturing costs may increase compared to the cosmetic effect.

Further, the cosmetic composition of the present disclosure may be used for pets depending on the formulation. For example, it may be prepared in various forms such as solution, solvent gel, emulsion, oil, wax, aerosol, etc., such as pet shampoo and pet rinse, and it may be prepared by adding a neutral detergent that is less irritating to the pet's skin and has excellent moisturizing power.

Further, the present disclosure has a technical feature in that it alleviates the cytotoxicity of the antibacterial peptide consisting of the amino acid sequence represented by SEQ ID NO: 1 and increases its antibacterial and antifungal activity and anti-biofilm activity.

Therefore, the present disclosure provides a method for inhibiting bacteria or fungi, the method including the step of treating an individual with a nanoparticle in which an antibacterial peptide consisting of the amino acid sequence represented by SEQ ID NO: 1 or a fragment thereof is encapsulated with chitosan.

The method of inhibiting bacteria or fungi of the present disclosure may be performed by treating an individual with a nanoparticle in which an antibacterial peptide consisting of the amino acid sequence represented by SEQ ID NO: 1 or a fragment thereof is encapsulated with chitosan.

In the present disclosure, the individual includes both biological and non-biological objects, and is not limited to any individual requiring the death of Acinetobacter.

The nanoparticles are characterized by showing antibacterial activity against Acinetobacter baumannii and antifungal activity against Candida albicans.

According to still another embodiment of the present disclosure, nanoparticles (octominin-CNP) in which an antibacterial peptide consisting of the amino acid sequence represented by SEQ ID NO: 1 is encapsulated with chitosan according to the present disclosure alleviated the cytotoxicity of octominin itself. Further, according to still another embodiment, as a result of treating A. baumannii and C. albicans with octominin-CNP, it was confirmed that pores were formed on the surface of the entire cell and cell contraction was caused, resulting in serious damage so that it has superior safety, antibacterial and antifungal activity than octominin itself.

Thus, the present disclosure provides a method for inhibiting a biofilm, the method including the step of treating a nanoparticle in which an antibacterial peptide consisting of the amino acid sequence represented by SEQ ID NO: 1 or a fragment thereof is encapsulated with chitosan.

The method of inhibiting a biofilm of the present disclosure may be performed by treating an individual with a composition for inhibiting a biofilm, including a nanoparticle in which an antibacterial peptide consisting of the amino acid sequence represented by SEQ ID NO: 1 or a fragment thereof is encapsulated with chitosan.

The nanoparticles are characterized in that they have biofilm formation inhibitory ability and biofilm eradication ability. The biofilm may be formed by one or more strains preferably selected from the group consisting of Acinetobacter baumannii and Candida albicans.

In the present disclosure, the “individual” include all biological or non-biological objects that have or are expected to form biofilms on its surface by one or more strains selected from the group consisting of Acinetobacter baumannii and Candida albicans.

A more detailed description of the composition for inhibiting biofilm is as described above.

Further, the present disclosure provides a method for preventing or treating a disease caused by infection with any one strain selected from the group consisting of Acinetobacter baumannii and Candida albicans, the method including the step of treating an individual with a nanoparticle in which an antibacterial peptide consisting of the amino acid sequence represented by SEQ ID NO: 1 or a fragment thereof is encapsulated with chitosan.

The method of preventing or treating a disease of the present disclosure may be performed by administering an individual with a pharmaceutical composition including a nanoparticle in which an antibacterial peptide consisting of the amino acid sequence represented by SEQ ID NO: 1 or a fragment thereof is encapsulated with chitosan.

In the present disclosure, the “individual” means mammals such as horses, sheep, pigs, goats, and dogs, including humans, birds, fish, crustaceans, reptiles, amphibians, etc., who may show a therapeutic effect against a disease infected with any one strain selected from the group consisting of Acinetobacter baumannii and Candida albicans, and preferably means humans.

Further, the present disclosure provides a food composition including a nanoparticle in which an antibacterial peptide consisting of the amino acid sequence represented by SEQ ID NO: 1 or a fragment thereof is encapsulated with chitosan.

Further, the present disclosure provides a cosmetic composition including a nanoparticle in which an antibacterial peptide consisting of the amino acid sequence represented by SEQ ID NO: 1 or a fragment thereof is encapsulated with chitosan.

A more detailed description of the pharmaceutical composition, food composition and cosmetic composition is as described above.

Further, the present disclosure provides a method for producing an antibacterial or antifungal nanoparticle in which an antibacterial peptide consisting of the amino acid sequence represented by SEQ ID NO: 1 or a fragment thereof is encapsulated with chitosan, the method including the step of mixing an antibacterial peptide consisting of the amino acid sequence represented by SEQ ID NO: 1 or a fragment thereof, chitosan and a chitosan derivative.

The chitosan derivative may be any one selected from the group consisting of carboxymethylcellulose (CMC), carboxymethyl chitosan (CMCS) and chitosan succinate, but is not limited thereto. In one embodiment of the present disclosure, carboxymethylcellulose (CMC) is used.

More specifically, the preparing method may include the following steps:

preparing a first mixture by mixing an antimicrobial peptide consisting of the amino acid sequence represented by SEQ ID NO: 1 or a fragment thereof and a chitosan derivative and mixing the first mixture with chitosan.

Furthermore, the present disclosure derived an optimal mixing ratio based on the encapsulation efficiency (EE %) and minimum particle size of the nanoparticles.

In the present disclosure, the antibacterial peptide consisting of the amino acid sequence represented by SEQ ID NO: 1 or fragment thereof, chitosan and a chitosan derivative may be mixed in a ratio (w/w/w) of 1 to 1.5:0.2 to 0.5:2. Preferably, they may be mixed in a ratio of 1 to 1.5:0.4:2 (w/w/w), and more preferably in a ratio of 1:0.4:2 (w/w/w).

The antibacterial or antifungal nanoparticles prepared by mixing at the above ratio showed significantly better encapsulation efficiency than the nanoparticles prepared by mixing at other ratios, so the antibacterial or antifungal nanoparticles prepared through the preparing method of the present disclosure may have more improved functionality.

The above-described contents of the present disclosure are equally applied to each other unless contradictory to each other, and implementation with appropriate changes by a person skilled in the art is also included in the scope of the present disclosure.

Hereinafter, the present disclosure is described in detail through Examples, but the scope of the present disclosure is not limited only to the following Examples.

### EXAMPLE 1. DERIVATION OF OPTIMAL MIXING RATIO FOR PREPARING OCTOMININ-CHITOSAN NANOPARTICLE (OCTOMININ-CNP)

Octominin-CNPs in which octominin, an antibacterial peptide consisting of the amino acid sequence represented by SEQ ID NO: 1, is encapsulated with chitosan are obtained by ion gelation method using chitosan (CS), carboxymethyl chitosan (CMC) and octominin.

First, a 1 mg/mL chitosan solution was prepared by dissolving chitosan in 0.25% (v/v) acetic acid (pH 6). In addition, a CMC solution (1 mg/mL) was prepared at pH 7.4 using distilled water. Both the CS and CMC solutions were filtered using a 1 μm syringe filter (Sartorius, Goettingen, Germany). Octominin was prepared by dissolving it at a concentration of 1 mg/mL in nuclease-free water.

Then, for the preparation of octominin-CNPs, the octominin prepared above was added to CMC according to the ratio shown in Table 1 below.

TABLE 1

| | Reaction | Reaction | Reaction | Reaction | Reaction |
|---|---|---|---|---|---|
| | 1 | 2 | 3 | 4 | 5 |
| CS (mg) | 0.4 | 0.4 | 0.4 | 0.4 | 0.4 |
| Octominin (mg) | 0 | 0.25 | 0.5 | 1.0 | 1.5 |
| CMC (mg) | 2.0 | 2.0 | 2.0 | 2.0 | 2.0 |
| H₂O (mL) | 1.5 | 1.25 | 1 | 0.5 | 0 |
| Total reaction | 3.9 | 3.9 | 3.9 | 3.9 | 3.9 |
| volume (mL) | | | | | |

The respective mixtures were continuously mixed on a magnetic stirrer for 45 minutes. Then, distilled water was added to equalize the volume of each reaction mixture, and 0.4 mL of the chitosan solution was added dropwise to each mixture for 1 minute while continuously stirring. The final mixture was stirred for an additional 2 hours. Octominin-encapsulated chitosan nanoparticles (octominin-CNP) were collected by centrifugation at 4° C. at 12000 rpm for 30 minutes. CNPs isolated from the supernatant were suspended in 1× phosphate-buffered saline (PBS). The supernatant from which CNP was separated was collected, and the residual peptide concentration was measured using Nanodrop (Thermo Scientific, Massachusetts, USA). Encapsulation efficiency (EE %) and loading capacity (LC %) were calculated through Equation 1 or Equation 2 below.

$$EE \% = \frac{( Initial weight of Octominin - Remained Octominin weight in supernetant )}{Initial weight of the Octominin} \times 100 \% \qquad [ Equation 1 ]$$

$$LC = \frac{( Initial weight of Octominin - Remained Octominin weight in supernetant )}{T0tal weight of the chitosan used for the encapsulation} \qquad [ Equation 2 ]$$

The optimal mixing ratio for encapsulation was determined by identifying the EE %, LC %, and minimum particle size of the octominin-CNP calculated as above.

TABLE 2

| | Reaction 1 | Reaction 2 | Reaction 3 | Reaction 4 | Reaction 5 |
|---|---|---|---|---|---|
| CS: | 0.4:0:2 | 0.4:0.25:2 | 0.4:0.5:2 | 0.4:1:2 | 0.4:1.5:2 |
| Octominin: | | | | | |
| CMC | | | | | |
| Particle size | 235.10 ± | 552.86 ± | 364.57 ± | 185.63 ± | 237.60 ± |
| (nm) | 2.48 | 19.24 | 11.78 | 10.53 | 14.37 |
| EE % | NA | 94.8 | 94.8 | 96.49 | 78.16 |
| LC % | NA | 9.88 | 19.75 | 40.20 | 48.85 |

As a result, as shown in Table 2 above, reaction mixture 4 (Reaction 4) having a chitosan:octominin:CMC ratio of 0.4:1:2 w/w/w indicated the highest EE % (96.49%) and the lowest minimum particle size (185.63 nm). Reaction mixture 5 showed a higher loading capacity of 48.85% compared to the loading capacity of reaction mixture 4 (40.20%), but a lower EE % (78.16%). Therefore, it was confirmed that the optimal mixing ratio was chitosan:octominin:CMC=0.4:1:2 w/w/w in the mixing ratio of octominin-CNP based on the EE % and the minimum particle size.

Therefore, octominin-CNP prepared by mixing chitosan, octominin, and CMC at a ratio of 0.4:1:2 was used in subsequent experiments. In addition, instead of the mixing process of octominin and CMC, 1 mL of water was mixed with 1 mL of CMC to prepare chitosan-nanoparticles (CNP) without octominin encapsulation, which was used as a control.

### EXAMPLE 2. CONFIRMATION OF OCTOMININ-CNP'S PROPERTY

### 2-1. Analysis Method

The size distribution and zeta potential of octominin-CNP and CNP were measured using a Malvern Zetasizer Nano-ZS ZEN 3600 (Malvern Panalytical, Cambridge, UK). In addition, the morphological property of octominin-CNPs and CNPs was determined through transmission electron microscopy (TEM) and emission scanning electron microscopy (FE-SEM) analysis.

First, after dissolving octominin-CNPs or CNPs in PBS for TEM analysis, 10 μl of it was placed on a formvar- and carbon-coated copper grid disk, and incubation was performed for 10 minutes. Excess samples were eradicated using filter paper. Then, 5 μl of 4% uranyl acetate (Sigma-Aldrich, Darmstadt, Germany) was placed on a grid for less than 5 seconds, and then excess uranyl acetate was eradicated by suction using a filter paper. The dried grids were observed and imaged with a 300 keV field emission-transmission electron microscope (Tecnai™ G2 F30 super-twin (FEI), (Oregon, USA)).

Further, the dried octominin-CNPs and CNPs were coated with platinum through ion sputtering (E-1030, (Hitachi, Japan)), and they were observed with FE-SEM (Sirion FEI, (Eindhoven, Netherlands)).

In addition, octominin-CNPs or CNPs were dissolved in 1 mL of 1× PBS and mild-sonicated t identify the release profile. The dissolved octominin-CNPs or CNPs were placed on a rocker at 18 RPM for 24 hours at room temperature (25±3° C.). Thereafter, the octominin-CNPs or CNPs were centrifuged at 4° C. at 12000 rpm for 30 minutes to remove the supernatant, and the concentration of octominin was measured.

The precipitated octominin-CNPs or CNPs were dissolved in 1 mL of 1× PBS by mild sonication and placed on a rocker under the same conditions as above. The concentration of octominin was measured repeatedly every 24 hours, and the amount of octominin released at each time point was calculated.

The above respective experiments were performed using three octominin-CNPs and CNPs prepared under the same conditions, and the values of the respective experimental groups were averaged.

### 2-2. Analysis Result

The properties and release kinetics of octominin-CNP prepared according to the present disclosure were identified. The dynamic light scattering analysis results of octominin-CNPs and CNPs are shown in Table 3 below.

TABLE 3

| | | Mean particle | | Zeta | | |
|---|---|---|---|---|---|---|
| Formulation | CS:CMC:AMP | size (mm) | [symbol] | potential (mV) | EE % | LC % |
| CNPs | 0.4:2:0 | 246. [symbol] ± 1.98 | 0.14 ± 0.005 | [symbol] ± [symbol] | NA | NA |
| Octominin-CNPs | 0.4:2:1 | 372. [symbol] ± 2.31 | 0.24 ± 0.009 | [symbol] ± [symbol] | [symbol] .6% | [symbol] 7.4% |

[symbol]

indicates data missing or illegible when filed

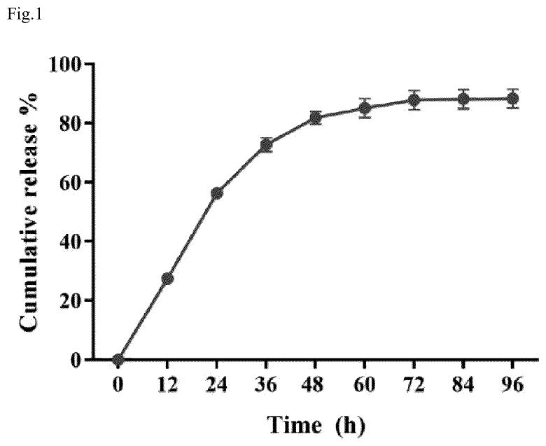

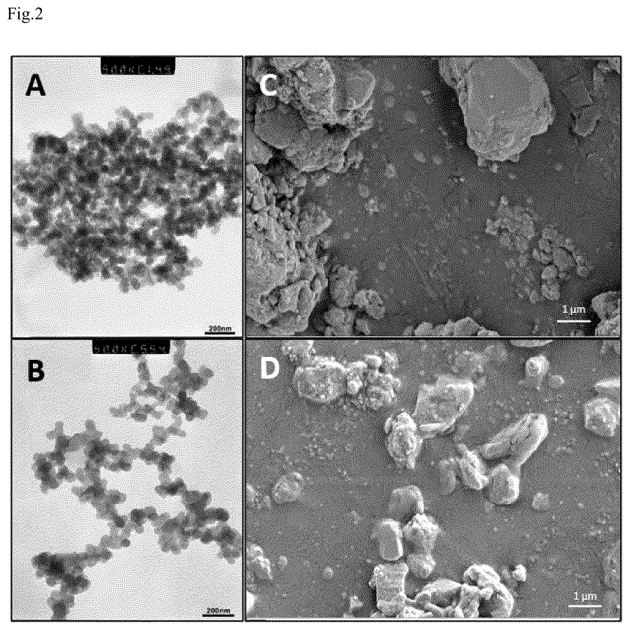

It was confirmed that the diameters of octominin-CNPs and CNPs were 372.8±2.3 nm and 246.8±1.98 nm on average, respectively. Thus, it was confirmed that octominin-CNPs were slightly larger than CNPs in which octominin was not encapsulated. In addition, for octominin-CNP and CNP, the cationic nature of CS in PBS at pH 7.4 were positive zeta potential of +51.23±0.38 mV and +59.33±3.63 mV, respectively, for the two NPs. In addition, for octominin-CNP, in order to quantify the octominin remaining in the supernatant, EE % and LC % were calculated in the same manner as in Example 1. As a result, the EE % of octominin-CNP was 93.6%, and the LC % was 37.4%. Further, the peptide (octominin) release kinetics of octominin-CNP for 96 hours at pH 7.4 using PBS were confirmed and shown in FIG. 1. The initial peptide cumulative release profile showed a gradual linear release up to 56.2% by 24 hours, but later the release rate decreased, reaching a maximum cumulative release of 88.26% at 96 hours. Further, the morphological properties of octominin-CNPs and CNPs were confirmed using TEM and FE-SEM, and the results are shown in FIG. 2. FIG. 2, panel A is a TEM image of CNPs, and FIG. 2, panel B is a TEM image of octominin-CNPs. Further, FIG. 2, panel C is a FE-SEM image of CNPs, and FIG. 2, panel D is a FE-SEM image of octominin-CNPs. As shown in FIG. 2, it was confirmed from the TEM micrograph that both octominin-CNPs and CNPs had rounded corners. As a result of FE-SEM analysis, both octominin-CNPs and CNPs showed irregularly shaped particles due to the possibility of particle aggregation, and octominin-CNPs showed a relatively low level of aggregation compared to CNPs.

### EXAMPLE 3. CONFIRMATION OF CYTOTOXICITY OF OCTOMININ-CNPS OR FREE OCTOMININ

### 3-1. Analysis Method

After treating HEK293 cells with octominin-CNP and octominin, MTT (3-(4,5-dimethylthiazol-2-yl)-2,5-diphenyltetrazolium bromide) assay was performed to confirm cell viability.

First, HEK293 cells were cultured using DMEM (Dulbecco's modified Eagle's medium (Sigma-Aldrich, Munich, Germany)) containing 10% (v/v) fetal bovine serum (FBS, (Sigma-Aldrich, Munich, Germany)) and antibiotic-antimycotic (Thermo Fisher Scientific, Waltham, MA, USA). In addition, it was cultured for 24 hours under humidified conditions of 5% CO₂ and 37° C. The cells were collected and seeded in a 96-well microplate at a cell density of 2.0×105 cells/mL (100 μL/well) and allowed to adhere to the well for 12 hours. The culture medium was then replaced, and they were treated with octominin-CNP (0 to 400 μg/mL) or octominin (0 to 400 μg/mL). After 24 hours of culture, the culture medium was replaced with fresh medium (90 μL), and 10 μL of MTT (5 μg/mL) was added to each well, followed by incubation at 37° C. for 4 hours. Then, 50 μL of DMSO was added to the wells in which the culture medium was eradicated to solubilize the resulting formazan dye. Their absorbances were measured at 595 nm using a microplate spectrophotometer (Bio-Rad, Saint Louis, (MO, USA)).

### 3-2. Analysis Result

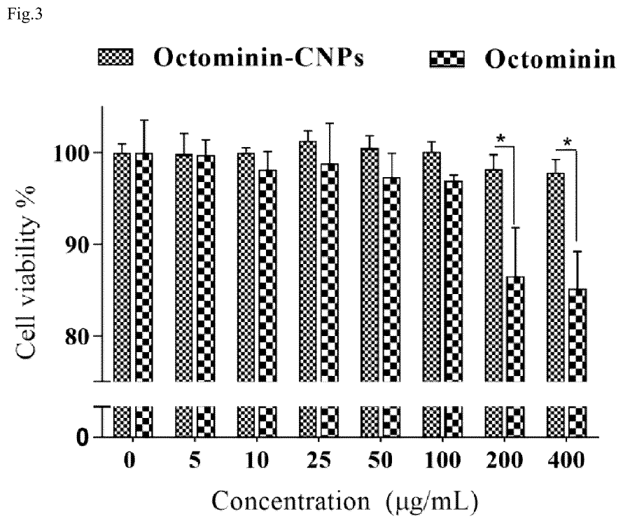

As a result of confirming the cytotoxicity of octominin-CNPs and free octominin on HEK 293 cells, as shown in FIG. 3, it was confirmed that octominin-CNPs and free octominin reduced minimum cellular viability up to a peptide concentration of 100 μg/mL. However, when the concentration of octominin was increased (200 and 400 μg/mL), a significant decrease in viability was observed in the experimental group treated with free octominin compared to octominin-CNPs (p<0.05). Further, octominin-CNPs showed 97.38% viability, whereas free octominin showed 85.19% viability at the highest concentration (400 μg/mL). Therefore, it was confirmed that octominin-CNP mitigates the cytotoxicity of octominin itself and has better cytocompatibility than octominin itself.

### EXAMPLE 4. CONFIRMATION OF ANTIBACTERIAL AND ANTIFUNGAL ACTIVITIES OF OCTOMININ-CNPS OR FREE OCTOMININ

### 4-1. Analysis Method

Prior to confirming the antibacterial and antifungal activity of octominin-CNP or free octominin, for the antibacterial activity of octominin against Acinetobacter baumannii (A. baumannii) through preliminary analysis, it was confirmed that minimum inhibitory concentration (MIC) was 5 μg/mL, and the minimum bactericidal concentration (MBC) was 10 μg/mL. In addition, for the antifungal activity against Candida albicans (C. albicans), it was confirmed that the MIC was 50 μg/mL and the minimum fungicidal concentration (MFC) was 200 μg/mL. Accordingly, the antibacterial and antifungal activities of octominin-CNP and free octominin against A. baumannii and C. albicans were confirmed at the MIC and MBC/MFC peptide concentrations.

First, A. baumannii was cultured at 25° C. using tryptic soy broth/agar media. Further, in order to identify antifungal activity, C. albicans was cultured at 37° C. using potato dextrose broth/agar media. The treatment concentration of octominin-CNP for each strain was calculated based on the amount of encapsulated octominin. Microdilution susceptibility tests were performed to determine time kill kinetic analysis according to the M07-A guidelines of the Clinical and Laboratory Standards Institute (CLSI). A. baumannii and C. albicans prepared as described above were seeded in triplicate at a density of 1×106 CFU/mL in a 96-well microplate (190 μL/well). Accordingly, 10 μL of octominin-CNP and 10 μL of octominin were treated in different concentrations (0 to 300 μg/mL for C. albicans and 0 to 50 μg/mL for A. baumannii) in each well and incubated for 24 hours. The growth of each strain was measured in an optical density at 595 nm (OD₅₉₅) at 3-hour intervals (0, 3, 6, 9, 12, 15, 18, 21 and 24 hours).

### 4-2. Analysis Result

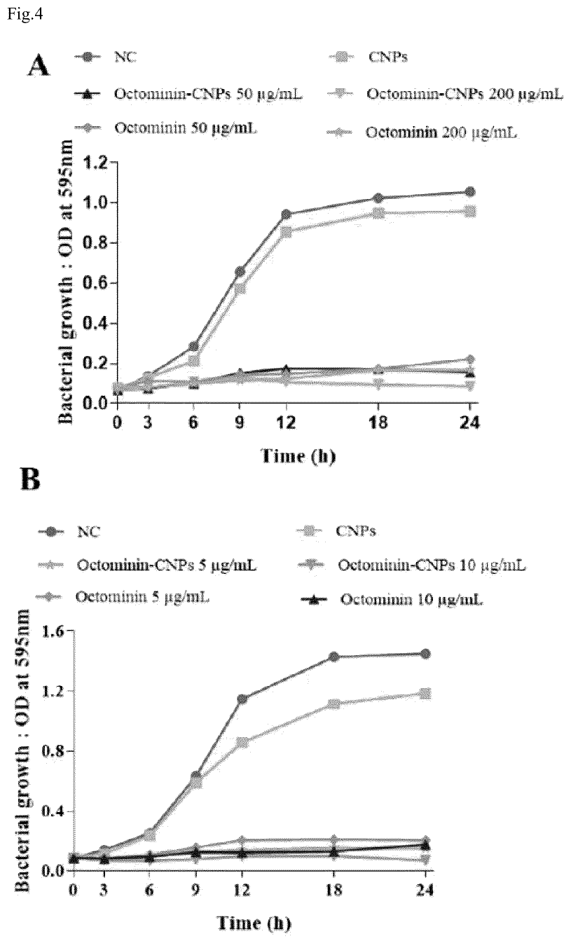

FIG. 4 shows the time-kill kinetic activity results according to the treatment of octominin-CNP and free octominin at each concentration. FIG. 4, panel A shows the result for C. albicans, and FIG. 4, panel B shows the result for A. baumannii. As a result, the experimental group treated with free octominin showed higher antibacterial and antifungal activity than the octominin-CNP in the initial stage. However, at the later stage, octominin-CNP showed better antibacterial and antifungal activity than free octominin (See 200 μg/mL treatment group of FIG. 4, panel A and 10 μg/mL treatment group of FIG. 4, panel B).

### EXAMPLE 5. CONFIRMATION OF CELL MORPHOLOGICAL CHANGES ACCORDING TO OCTOMININ-CNP OR FREE OCTOMININ TREATMENT

### 5-1. Analysis Method

After treating A. baumannii and C. albicans with octominin-CNP or octominin, ultra-structural changes were confirmed. Each strain was cultured under the same conditions as in Example 4, and each strain was used at a density of 1×106 CFU/mL. C. albicans was treated with 50 μg/mL of octominin-CNPs and 200 μg/mL of octominin and incubated at 37° C. for 9 hours. A. baumannii was treated with 5 μg/mL of octominin-CNPs and 10 μg/mL of octominin and incubated at 25° C. for 9 hours. Each strain was treated with PBS as a negative control, C. albicans was treated with fluconazole, and A. baumannii was treated with chloramphenicol. Then, cells were collected by centrifugation at 1500×g for 10 minutes, washed with PBS, and pre-fixed with 2.5% glutaraldehyde for 20 minutes. After washing with PBS, the samples were dehydrated by washing with serial dilutions of ethanol (30%, 50%, 70%, 80%, 90%, and 100%). Platinum coating was performed through ion sputtering (E-1030, (Hitachi, Japan)), and the samples were observed with FE-SEM (Sirion FEI, (Eindhoven, Netherlands)).

### 5-2. Analysis Result

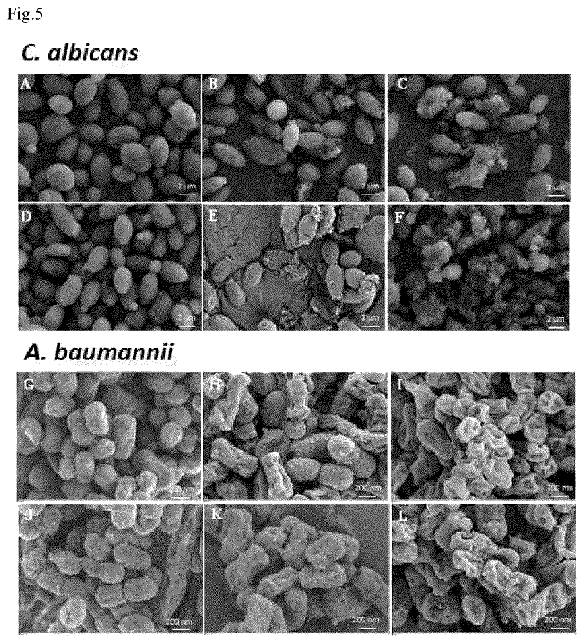

As a result of confirming morphological changes after treatment of octominin-CNP and free octominin to C. albicans and A. baumannii, it was confirmed that when C. albicans was treated with CNP (FIG. 5, panel D), no change was caused on the surface of the fungal cells, and they had the same morphological structure as the negative control group (FIG. 5, panel A). However, it was confirmed that when treated with octominin-CNP or free octominin, the surface of the fungal cells was damaged, and in particular, the degree of damage in the experimental groups treated with octominin-CNP (FIG. 5, panel E and FIG. 5, panel F) was stronger than that in the experimental groups treated with the free octominin (FIG. 5, panel B and FIG. 5, panel C). Specifically, it was confirmed that small pores were formed in the experimental group treated with MIC (50 μg/mL) of free octominin, resulting in slight damage, and cell contraction was caused along with pore formation, resulting in severe damage in the octominin-CNP (50 μg/mL)-treated experimental group. Further, it was confirmed that the experimental group treated with free octominin with MBC (200 μg/mL) caused cell contraction and cell destruction in a small number of cells, but the experimental group treated with octominin-CNP (200 μg/mL) caused total cell destruction and caused great damage to the fungal cells. A similar morphological change pattern was identified in A. baumannii.

In the case of A. baumannii, unlike C. albicans, weak cell surface contraction was observed by CNP treatment (FIG. 5, panel J), but it was not significant. The experimental group treated with octominin-CNPs showed superior activity against A. baumannii at the MIC (5 μg/mL) and MBC (10 μg/mL) levels compared to the experimental groups treated with free octominin at the same concentration.

Specifically, in the case of MIC concentration treatment, it was confirmed that the number of cells in which cell contraction occurred was higher in the experimental group treated with octominin-CNP (FIG. 5, panel K) than in the experimental group treated with free octominin (FIG. 5, panel H). In the case of MBC concentration treatment, both experimental groups (FIG. 5, panel I and FIG. 5, panel L) showed hole formation and bacterial cell damage, but the degree thereof was more severe in the experimental group treated with octominin-CNP (FIG. 5, panel L).

### EXAMPLE 6. CONFIRMATION OF CELL MEMBRANE PERMEABILITY CHANGE AND GENERATION OF REACTIVE OXYGEN SPECIES (ROS) ACCORDING TO OCTOMININ-CNP OR FREE OCTOMININ TREATMENT

### 6-1. Analysis Method

After treating A. baumannii and C. albicans with octominin-CNP or octominin, cell membrane permeability change and ROS production were confirmed through analysis of propidium iodide (PI) and H₂DCF-DA(2′,7′-dichlorodihydrofluorescein diacetate) staining combined with FDA (fluorescein diacetate) staining. Each strain was prepared at a cell density of 1×106 CFU/mL, C. albicans was treated with 50 μg/mL of octominin and 200 μg/mL of octominin-CNP, and A. baumannii was treated with 5 μg/mL of octominin and 10 μg/mL of octominin-CNP. Negative control and CNPs treatment were performed, and they were incubated for 12 hours. Then, each strain was obtained by centrifugation at 1500×g for 10 minutes. The isolated cell pellet was washed with PBS and resuspended in PBS. To monitor permeability changes, 1 mL of the respective suspensions was stained with 50 μg/mL of PI (Sigma Aldrich, Saint Louis, USA) or 40 μg/mL of FDA (Sigma Aldrich, Saint Louis, USA) and incubated for 30 minutes in the dark. To confirm ROS production, 1 mL of the respective cell suspensions was stained with 50 μg/mL of H₂DCF-DA and incubated for 30 minutes in the dark. Thereafter, excess dye was removed by centrifugation at 1500×g and washed three times with PBS. The cell pellet was resuspended in 20 μL of PBS, and 5 μL of the suspension was placed on a glass slide and observed under a confocal laser scanning microscope (Carl Zeiss, Jena, Germany). In cell monitoring, dead cells or cells with altered membrane permeability were identified by red fluorescence, and live cells were identified by green fluorescence. Regarding ROS generation, green fluorescence was confirmed through H₂DCF-DA staining. Excitation and emission wavelengths for red fluorescence were 535 and 617 nm, respectively, and excitation and emission wavelengths for green fluorescence were 488 and 353 nm, respectively.

### 6-2. Analysis Result

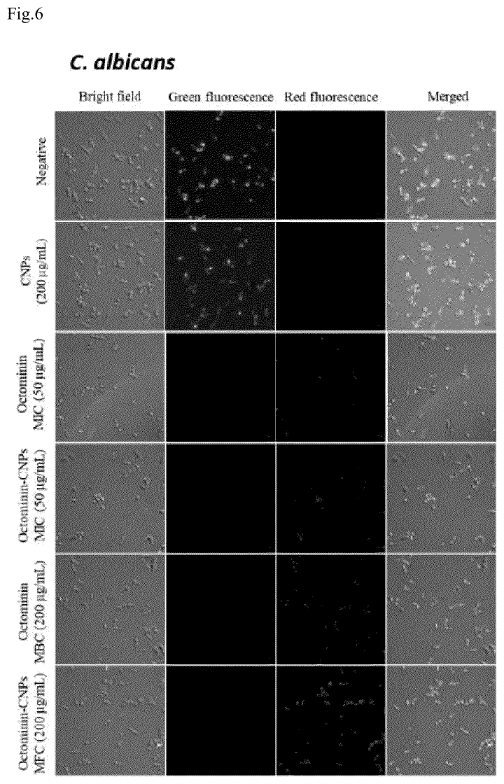

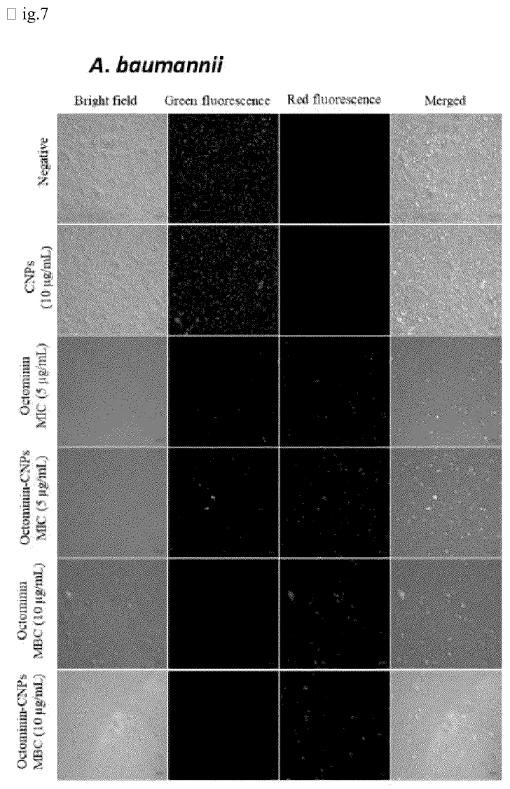

PI penetrates cells with altered membrane permeability to produce red fluorescence, and FDA generates green fluorescence in viable cells. Using this, changes in membrane permeability of cells treated with octominin-CNP or free octominin were confirmed and shown in FIGS. 6 and 7. As a result, since only green fluorescence was expressed in the experimental group treated with CNP, membrane permeability was not changed. However, in the case of C. albicans and A. baumannii treated with octominin-CNP or free octominin at MIC concentrations, red fluorescent cells were observed and green fluorescent cells were observed at low levels. In addition, when octominin-CNP and free octominin were treated at the MBC level, only red fluorescence without green fluorescence was observed. Therefore, it was confirmed that both free octominin and octominin-CNP may change cell membrane permeability.

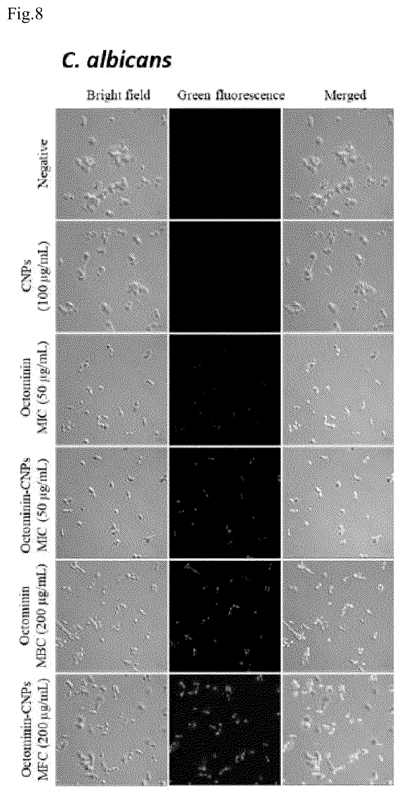

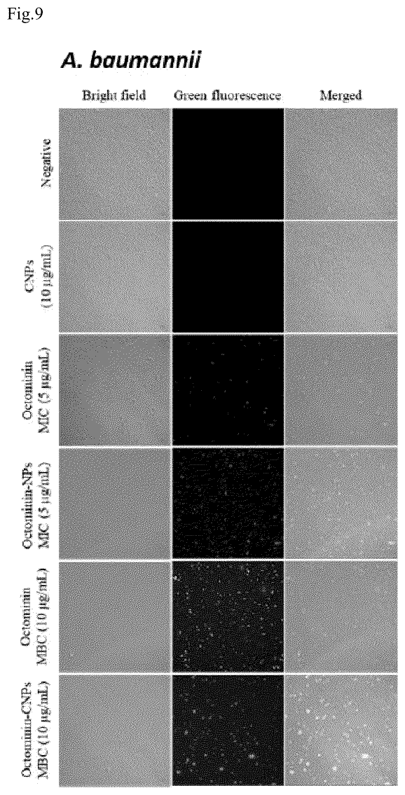

Further, C. albicans and A. baumannii treated with octominin-CNP or free octominin were stained with H₂DCFDA, and the ROS generating ability was confirmed. The results are shown in FIGS. 8 and 9. When the experimental group treated with MIC and MFC/MBC levels of octominin-CNP in C. albicans and A. baumannii was compared with the experimental group treated with free octominin at MIC and MFC/MBC levels, significantly higher green fluorescence was observed. Therefore, it was confirmed through this that octominin-CNP induces higher ROS production than free octominin.

### EXAMPLE 7. CONFIRMATION OF ANTI-BIOFILM ACTIVITY ACCORDING TO OCTOMININ-CNP AND FREE OCTOMININ TREATMENT

### 7-1. Analysis Method

In order to confirm the inhibitory and eradication activities of octominin-CNP and octominin on biofilms of A. baumannii and C. albicans, a biofilm quantification method based on crystal violet staining was performed. To confirm the biofilm inhibitory activity, each strain was prepared at a cell density of 1×106 CFU/mL, C. albicans was treated with 50 μg/mL of octominin and 200 μg/mL of octominin-CNP, and A. baumannii was treated with 5 μg/mL of octominin and 10 μg/mL of octominin-CNP. Negative control and CNPs treatment were performed, and they were incubated for 24 hours.

For the biofilm eradication assay, initially, 1×106 CFU/mL of A. baumannii and C. albicans were placed in a 96 microwell plate (200 μL/well) and cultured for 20 hours to induce biofilm formation. The supernatant was removed, the biofilm was washed with PBS, and the medium in each well was replaced. Accordingly, C. albicans was treated with 50 μg/mL of octominin and 200 μg/mL of octominin-CNP, and A. baumannii was treated with 5 μg/mL of octominin and 10 μg/mL of octominin-CNP. Negative control and CNPs treatment were performed, and they were incubated for 24 hours. Then, in order to confirm the remaining biofilm, the plate for the biofilm inhibition assay and the biofilm eradication assay was stained with crystal violet. Initially remaining supernatant was eradicated, and the biofilm was washed using PBS. Then, the biofilm was fixed for 10 minutes using 100% methanol. Then, methanol was eradicated, and the biofilm was stained with 0.1% (w/v) crystal violet (CV, (Sigma-Aldrich, USA)) for 30 minutes at room temperature (26±2° C.). The biofilm was washed three times with PBS to eradicate excess CV. Finally, 95% ethanol was added to the plate and shaken to dissolve the CV-stained biofilm. Its absorbance was measured at 595 nm using a microplate spectrophotometer. The biofilm inhibition/eradication ratio was calculated through Equation 3 below.

Inhibition or eradication of biofilm formation %=[1−(Ab test/Ab negative control)]×100% [Equation 3]

In Equation 3, Ab test represents the absorbance value of octominin or chloramphenicol, and Ab negative control represents the absorbance of the negative control group (PBS).

### 7-2. Analysis Result

After treating C. albicans and A. baumannii with octominin-CNP and free octominin, biofilm formation inhibition and biofilm eradication effects were confirmed through CV staining.

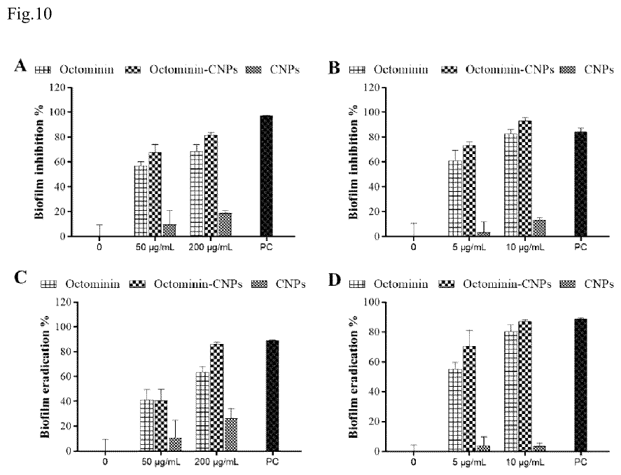

As a result, as shown in FIG. 10, octominin-CNP showed better biofilm formation inhibitory activity against C. albicans and A. baumannii than free octominin. Specifically, in C. albicans, octominin-CNP showed 67.97% of biofilm formation inhibitory activity at the MIC concentration and 82.28% of biofilm formation inhibitory activity at the MBC level. On the other hand, free octominin exhibited 57.12% and 68.91% of biofilm formation inhibitory activity, respectively, at the same concentration (FIG. 10, panel A).

Further, in A. baumannii, octominin-CNP showed 73.70% of biofilm formation inhibitory activity at MIC concentration and 93.21% of biofilm formation inhibitory activity at MBC concentration. On the other hand, free octominin exhibited 61.30% and 83.06%, respectively, at the same concentration (FIG. 10, panel B).

Further, as a result of confirming the biofilm eradication effect, in C. albicans, octominin-CNP exhibited 40.73% eradication activity at the MIC concentration and 86.61% eradication activity at the MBC concentration. On the other hand, free octominin exhibited 41.50% and 63.57% eradication activity, respectively, at the same concentration (FIG. 10, panel C). Further, in A. baumannii, octominin-CNP exhibited 70.74% eradication activity at the MIC concentration and 87.30% eradication activity at the MBC concentration. On the other hand, free octominin exhibited 55.66% and 80.53% activity, respectively, at the same concentration (FIG. 10, panel D).

Therefore, it was confirmed that the octominin-CNP according to the present disclosure has a better anti-biofilm activity than octominin itself.

Overall, the present disclosure prepared octominin-chitosan nanoparticles in which octominin was encapsulated with chitosan (Octominin-chitosan nanoparticle) and derived an optimal mixing ratio thereof, and confirmed its antibacterial, antifungal and anti-biofilm activity against Acinetobacter baumannii and Candida albicans.

The octominin-CNP alleviates the cytotoxicity of octominin itself and causes a greater morphological change on the cell surface of A. baumannii and C. albicans than octominin itself, showing excellent antibacterial and antifungal activity as well as excellent biofilm formation inhibition and eradication effects, so that the octominin-CNP may be usefully used as a composition for antibacterial or biofilm formation inhibition including the same.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A method for inhibiting infection of an Acinetobacter sp. strain, the method comprising a step of treating an individual with an antimicrobial peptide consisting of an amino acid sequence represented by SEQ ID NO: 1 or a fragment thereof.

2. The method of claim 1, wherein the Acinetobacter sp. strain includes Acinetobacter baumannii.

3. A method for inhibiting a biofilm, the method comprising a step of treating an antimicrobial peptide consisting of an amino acid sequence represented by SEQ ID NO: 1 or a fragment thereof.

4. The method of claim 3, wherein the biofilm is formed by Acinetobacter baumannii.

5. The method of claim 3, wherein the antimicrobial peptide consisting of the amino acid sequence represented by SEQ ID NO: 1 or the fragment thereof has a biofilm formation inhibitory ability and biofilm eradication ability.

6. A method for preventing or treating an infectious disease by an Acinetobacter sp. strain, the method comprising a step of treating an individual with an antimicrobial peptide consisting of an amino acid sequence represented by SEQ ID NO: 1 or a fragment thereof.

7. A method for inhibiting bacteria or fungi, the method comprising a step of treating an individual with a nanoparticle in which an antibacterial peptide consisting of an amino acid sequence represented by SEQ ID NO: 1 or a fragment thereof is encapsulated with chitosan.

8. The method of claim 7, wherein the nanoparticle shows antibacterial activity against Acinetobacter baumannii.

9. The method of claim 7, wherein the nanoparticle shows antifungal activity against Candida albicans.

10. A method for inhibiting a biofilm, the method comprising a step of treating an individual with a nanoparticle in which an antibacterial peptide consisting of an amino acid sequence represented by SEQ ID NO: 1 or a fragment thereof is encapsulated with chitosan.

11. The method of claim 10, wherein the nanoparticle has a biofilm formation inhibitory ability and biofilm eradication ability.

12. A method for preventing or treating a disease caused by infection with any one strain selected from the group consisting of Acinetobacter baumannii and Candida albicans, the method comprising a step of treating an individual with a nanoparticle in which an antibacterial peptide consisting of an amino acid sequence represented by SEQ ID NO: 1 or a fragment thereof is encapsulated with chitosan.

13. A method for producing an antibacterial or antifungal nanoparticle in which an antibacterial peptide consisting of an amino acid sequence represented by SEQ ID NO: 1 or a fragment thereof is encapsulated with chitosan, the method comprising a step of mixing the antibacterial peptide consisting of the amino acid sequence represented by SEQ ID NO: 1 or the fragment thereof, the chitosan and a chitosan derivative.

14. The method of claim 13, wherein the chitosan derivative includes any one selected from the group consisting of carboxymethylcellulose (CMC), carboxymethyl chitosan (CMCS), and chitosan succinate.

15. The method of claim 13, wherein the mixing step includes the following steps:
preparing a first mixture by mixing the antimicrobial peptide consisting of the amino acid sequence represented by SEQ ID NO: 1 or the fragment thereof and the chitosan derivative, and
mixing the first mixture with the chitosan.

16. The method of claim 13, wherein the antibacterial peptide consisting of the amino acid sequence represented by SEQ ID NO: 1 or the fragment thereof, the chitosan and the chitosan derivative are mixed in a ratio (w/w/w) of 1 to 1.5:0.2 to 0.5:2.


SEQUENCE LISTING (1 sequence)

SEQ ID NO: 1 - protein, 23 aa, synthetic construct
  source 1..23: note = Octominin
GWLIRGAIHAGKAIHGLIHRRRH